(19) United States
(12) Patent Application Publication
(10) Pub. No.: US 2011/0189718 A1
(43) Pub. Date: Aug. 4, 2011

(54) Systems And Methods For Segregating Mixed Material Streams

(76) Inventors: Andrea Lee Whitson, Pace, FL (US); Thomas J.A. Campbell, Rockport, ME (US); Shuanghe Shi, Southborough, MA (US); Tyler Christian Hammond, Arlington, MA (US)

(21) Appl. No.: 12/982,859

(22) Filed: Dec. 30, 2010

Related U.S. Application Data
(60) Provisional application No. 61/291,228, filed on Dec. 30, 2009.

(51) Int. Cl. C12Q 1/02; B07C 5/00; F23L 7/00; F23G 5/00; C12M 1/00; C10L 1/10

(52) U.S. Cl. 435/29; 209/509; 209/10; 431/2; 110/346; 435/289.1; 44/300

(57) ABSTRACT

The invention relates to methods and systems of detecting useful material in a mixed solid, liquid and/or gaseous material stream. The methods include defining a value range requirement for at least one parameter of interest of useful material to be selected from the material stream, passing the material stream through at least one detector adapted to measure the parameter of interest of the material stream, and separating the material stream into useful material and residue based on the measured parameter. The systems comprise at least one detector adapted to measure a parameter of interest of the material stream passing therethrough; and at least one separator for separating the material stream into useful material and residue based on the measured parameter after passing through the detector. The system may further comprise treaters, processors, and controllers.

### REFERENCE TO RELATED APPLICATIONS

This patent application claims the benefit of U.S. Provisional Application No. 61/291,228 filed on Dec. 30, 2009, the disclosure of which is incorporated herein by reference in its entirety.

### BACKGROUND OF THE INVENTION

1. Field of the Invention

The present invention relates generally to the field of material handling and, more particularly, to systems and methods that can be used to detect and grade the desirable hydrocarbon material in bulk streams of mixed material.

2. Description of Related Art

Many different methods exist for reclaiming the energy in waste, including incineration, gasification and bio-digestion. However, the effectiveness and utility of these methods is often reduced through contamination of waste streams by hazardous materials that may contaminate the environment, and/or by the inclusion of materials in the waste stream that may have a negative impact on the process used to produce energy. For example, current physical sorting methods are not able to eliminate efficiently non-fuel components and contaminants, such as non-ferrous metals (e.g., lead and nickel-cadmium batteries) and halogenated plastics (e.g., PVC), that can produce dioxins when incinerated.

In order to optimize the reclamation of energy from the waste stream, it is necessary to remove from the stream materials such as contaminants, pollutants, and/or low energy materials that may negatively impact the efficiency of the process. Methods of analyzing the waste stream to detect contaminants, pollutants, and/or low-energy materials have generally been limited to detecting the presence or absence of a given material property. These methods are generally inefficient, at least because of the difficulties in accurately determining the properties of a material based purely on the presence or absence of a single material property, and also because such unsophisticated analyses are unable to ensure that all useful fuel material is collected for reuse while all other material is separated out.

There have been recent efforts to develop environmental friendly method and apparatus for sorting materials. U.S. Pat. No. 6,266,390 to Sommer, Jr. et al. (2001) discloses a system to sort materials using x-ray fluorescence. The system is limited in throughput rate due to the nature of the detection system. The x-ray fluorescence detector is not suitable for sorting out hydrocarbon materials for reuse as fuels from a complex waste stream.

Another effort is described in the PreGrant Publication no. US 2004/0066890 to Dalmijn et al. wherein the invention relates to a method and an apparatus for analyzing a flow of material using X rays. The method comprises radiating the material with at least two energy levels and measuring the transmission values to determine the thickness and composition of the material. However, the publication does not reveal how such determinations can be accomplished.

U.S. Pat. No. 7,099,433 to Sommer et al. (2006) discloses a metal sorting device including x-ray tube, a dual energy detector array. The device senses the presence of samples in the x-ray sensing region and initiates identifying and sorting the samples according to relative composition. The detection system based on one single material property may not be able to ensure that all useful material is separated from a complex waste stream. The disclosed apparatus is a metal sorting device.

Therefore it is necessary to have a method and a system that can separate a useful material, especially hydrocarbon material, from a solid, liquid and/or gaseous material stream complex waste stream based at least in part on measurements of at least one parameter of the material.

### SUMMARY OF THE INVENTION

The present invention is directed towards novel methods and systems for separating a useful material (e.g., a fuel) from a solid, liquid and/or gaseous material stream based at least in part on measurements of at least one parameter of the material, such as, but not limited to, effective atomic number (Z_{eff}) and/or density. The methods and systems can also be used to grade the algal fuel from live algae culture.

In one aspect, methods described herein may provide operators of energy reclamation plants with methods for developing easily determinable fuel specification criteria for incoming materials. Presently, incineration and gasification plant manufacturers are limited in the number and location of plants by their ability to reliably source acceptable fuels. They are not able to quantify a standard for the fuels and where there have been attempts to quantify a standard for the fuel, no continuous method has been employed for inspection to ensure compliance to that fuel standard. In general, these companies rely on training of human sorters and on random sampling of the fuel to ensure quality. The methods described herein allow for a quantifiable standard and for an ability to test to that standard automatically.

Moreover, the methods described herein can also be applied to live algae that is being grown in culture. The methods allow for grading the algal fuel and/or selecting individual organisms in the algal culture that have superior energy storing performance. The results of the screening can be used for selective breeding or to identify individual organisms whose genes should be harvested for future generations for genetically modified algae.

One aspect of the invention relates to a method of selecting useful material from a mixed material stream. The material stream may include a solid, liquid and/or gaseous material stream. The method includes the steps of defining a value range requirement for at least one parameter of interest of useful material to be selected from the material stream, passing the material stream through at least one detector adapted to measure the parameter of interest of the material stream, and separating the material stream into useful material and residue based on the measured parameter. The useful material may, for example, be a fuel such as, but not limited to, a hydrocarbon fuel material. Alternatively, useful material may include a material such as, but not limited to, metals such as iron, aluminum, mercury, and/or copper.

In one embodiment, the parameter of interest may be at least one of Z_{eff} and density. An upper limit for the density may be equal to the density of PVC, while an upper limit for Z_{eff} may be 8. The parameter of interest may include both Z_{eff} and density. At least one detector may be adapted to measure Z_{eff} and/or density of the material stream. The detector may be a dual-energy x-ray system. Alternatively, a first detector may be adapted to measure Z_{eff} of the material stream, with a second detector adapted to measure density of the material stream. The value range requirement of Z_{eff} and density may be selected to provide a useful material having a relatively high Gibbs free energy, Helmholz free energy, Higher Heating Value, or Lower Heating Value to carbon by weight ratio.

The method may further comprise processing the useful material to produce energy. The processing step may include at least one of incineration, gasification, or bio-digestion. In one embodiment, the method further comprises, prior to processing, treating the useful material to adjust a fuel performance property of the useful material. The treating step may include adding a high energetic material or a low energetic material to the useful material. The low energetic material may be added when the ratio of Z_{eff} to density of the useful material is greater than or equal to a first value, such as, but not limited to, a value of the ratio of Z_{eff} to density for cholesterol, but less than a second value, such as but not limited to, a value of the ratio of Z_{eff} to density for octane. The high energetic material may be added when the ratio of Z_{eff} to density of the useful material is greater than or equal to a second value, such as, but not limited to, a value of the ratio of Z_{eff} to density for octane.

In one embodiment, the treating step includes measuring the water content of the useful material, measuring a dual-energy transmissivity of the useful material, calculating an adjusted dual-energy transmissivity by compensating for the measured water content, and adjusting addition of high energetic material and low energetic material based at least in part thereon. The water content may be measured, for example, using a method based on microwave, mm-wave, and/or THz technology.

In one embodiment, the method further includes treating the residue based on a parameter of the residue measured by the detector. The parameter may include at least one of Z_{eff} and density.

Another aspect of the invention includes a system for selecting a useful material from a mixed solid, liquid or gaseous material stream passing through the system. The system includes at least one detector adapted to measure a parameter of interest of the material stream passing therethrough and at least one separator for separating the material stream into useful material and residue based on the measured parameter after passing through the detector. In one embodiment, the separator separates useful material from residue based on a value range requirement for the at least one measured parameter of interest. The system may further include at least one controller for controlling operation of at least one of the system, the detector, and the separator.

In one embodiment, the at least one detector is adapted to measure at least one of Z_{eff} and density of the material stream, and may be adapted to measure both Z_{eff} and density of the material stream. The at least one detector may include a dual-energy x-ray. The system may include a first detector adapted to measure Z_{eff} of the material stream and a second detector adapted to measure density of the material stream.

The system may include a processor for processing the useful material to produce energy. The processor may include at least one of an incinerator, a gasifier, or a bio-digestor. In one embodiment, the system includes at least one treater for treating the useful material to adjust a fuel performance property of the useful material prior to processing. The treater may add at least one of a high energetic material and a low energetic material to the useful material. The treater may be adapted to measure at least one of a water content of the useful material and a dual-energy transmissivity of the useful material. The water content is measured using at least one of microwave, mm-wave, and THz technology. In one embodiment, the system includes a residue treater for treating the residue separated from the useful material.

These and other objects, along with advantages and features of the present invention herein disclosed, will become more apparent through reference to the following description, the accompanying drawings, and the claims. Furthermore, it is to be understood that the features of the various embodiments described herein are not mutually exclusive and can exist in various combinations and permutations.

The more important features of the invention have thus been outlined in order that the more detailed description that follows may be better understood and in order that the present contribution to the art may better be appreciated. Additional features of the invention will be described hereinafter and will form the subject matter of the claims that follow.

Before explaining at least one embodiment of the invention in detail, it is to be understood that the invention is not limited in its application to the details of construction and the arrangements of the components set forth in the following description or illustrated in the drawings. The invention is capable of other embodiments and of being practiced and carried out in various ways. Also it is to be understood that the phraseology and terminology employed herein are for the purpose of description and should not be regarded as limiting.

As such, those skilled in the art will appreciate that the conception, upon which this disclosure is based, may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

The foregoing has outlined, rather broadly, the preferred feature of the present invention so that those skilled in the art may better understand the detailed description of the invention that follows. Additional features of the invention will be described hereinafter that form the subject of the claims of the invention. Those skilled in the art should appreciate that they can readily use the disclosed conception and specific embodiment as a basis for designing or modifying other structures for carrying out the same purposes of the present invention and that such other structures do not depart from the spirit and scope of the invention in its broadest form.

### BRIEF DESCRIPTION OF THE DRAWINGS

Other aspects, features, and advantages of the present invention will become more fully apparent from the following detailed description, the appended claim, and the accompanying drawings in which similar elements are given similar reference numerals.

In the drawings, like reference characters generally refer to the same parts throughout the different views. Also, the drawings are not necessarily to scale, emphasis instead generally being placed upon illustrating the principles of the invention. In the following description, various embodiments of the present invention are described with reference to the following drawings, in which:

FIG. 1 is a chart showing the relationship between Z_{eff} and density for various desirable fuels and non-fuel residue materials, in accordance with one embodiment of the invention;

FIG. 2 is a chart showing the relationship between Gibbs free energy and the ratio of Z_{eff} to density for various desirable fuels and non-fuel residue materials, in accordance with one embodiment of the invention;

FIGS. 3 and 4 depict dot product visualizations of example fuel materials against a salt representative vector, in accordance with one embodiment of the invention;

FIGS. 5 and 6 depict dot product visualizations of example fuel materials against a sugar representative vector, in accordance with one embodiment of the invention;

FIG. 7 is a chart depicting dot product visualizations for a blend of two example fuel materials in various concentrations against salt and sugar representative vectors, in accordance with one embodiment of the invention;

FIG. 8 is a chart showing a plot of sugar score against percentage fuel for an example fuel material, in accordance with one embodiment of the invention; and

FIG. 9 is a schematic view of a material handling system, in accordance with one embodiment of the invention.

### DESCRIPTION OF THE PREFERRED EMBODIMENT

Embodiments of the invention relate in general to improved methods, and associated apparatus and systems, for material handling, for example for facilitating process control for energy producers. The methods and systems described herein may be used, for example, to mine useful material from post-consumer waste, or be used to regulate the liberation of energy from algae producers, where digesters may be utilized to detect sugars in fuels coming into the process. Useful material may, for example, include, or consist essentially of, a fuel such as, but not limited to, a hydrocarbon fuel material. Alternatively, useful material may include a material such as, but not limited to, metals such as iron, aluminum, mercury, and/or copper. In alternative embodiments, methods and systems described herein may be used in gasification and incineration processes, for example to grade incoming fuel and optimally regulate the processes for fuels that do not come from what is traditionally thought of as the waste stream.

Example embodiments of the invention include methods, and associated apparatus and systems, for monitoring a solid, liquid, and/or gas material stream (e.g. a waste stream, a stream of coal slag, algae culture etc.) and segregating the material stream into material portions containing useful material (e.g., desirable fuels) and non-fuel residues (e.g., undesirable fuel materials, contaminants, etc.). These desirable fuel portions may then be used to generate usable energy through processes such as, but not limited to, incineration, gasification, and bio-digestion. In alternative embodiments, the stream of material may be monitored for other uses, such as, but not limited to, pollution monitoring, explosives detection, and/or mineral detection. The methods and systems described herein can also be applied to grade algal fuel from live algae culture. The results obtained can be used to select individual organisms in the algal culture that have superior energy performance for future generation of genetically modified algae.

Desirable hydrocarbon fuels generally reside within a particular range of density and Z_{eff}. By eliminating undesirable material from a material stream to leave only the material portions exhibiting the preferable Z_{eff}/density measurement of Z_{eff} and density for the material stream, processing of the resulting measurements using an appropriate comparison algorithm, and separation of the material into a fuel portion and a residue portion based at least in part on the results, the material stream can be processed to increase the efficiency of material used to generate energy. More particularly, while energy recycling from waste has many benefits, it is typically not a low carbon method of producing electricity. It is highly desirable to have a method that selects for fuels that have a higher ratio of Gibbs free energy, Helmholz free energy, Higher Heating Value, or Lower Heating Value to carbon by weight. Within the range of desirable hydrocarbon fuels, the measure of Z_{eff} and density has been determined to be an effective analog to the amount of energy that will be released per carbon atom, thereby allowing the fuel to be accurately selected from a material stream through measurement of Z_{eff} and density.

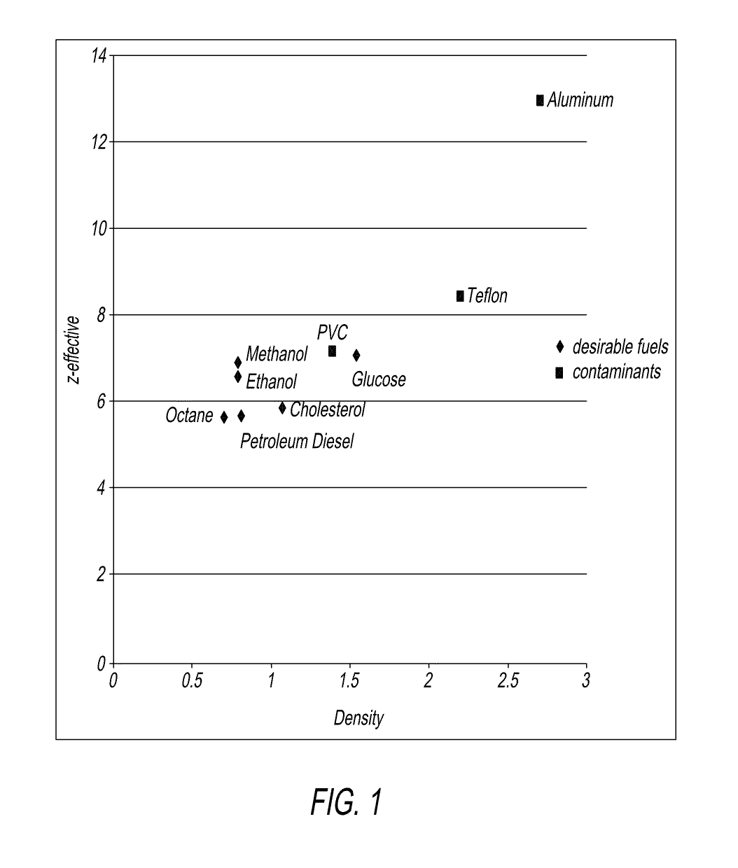

Example chart plotting Z_{eff} against density for a number of materials is shown in FIG. 1. Both of aluminum and Teflon having Z_{eff} of higher than 8 are contaminants. Methanol, ethanol, cholesterol, and petroleum diesel, octane are desirable fuels; they all have Z_{eff} of lower than 8 and density lower than PVC.

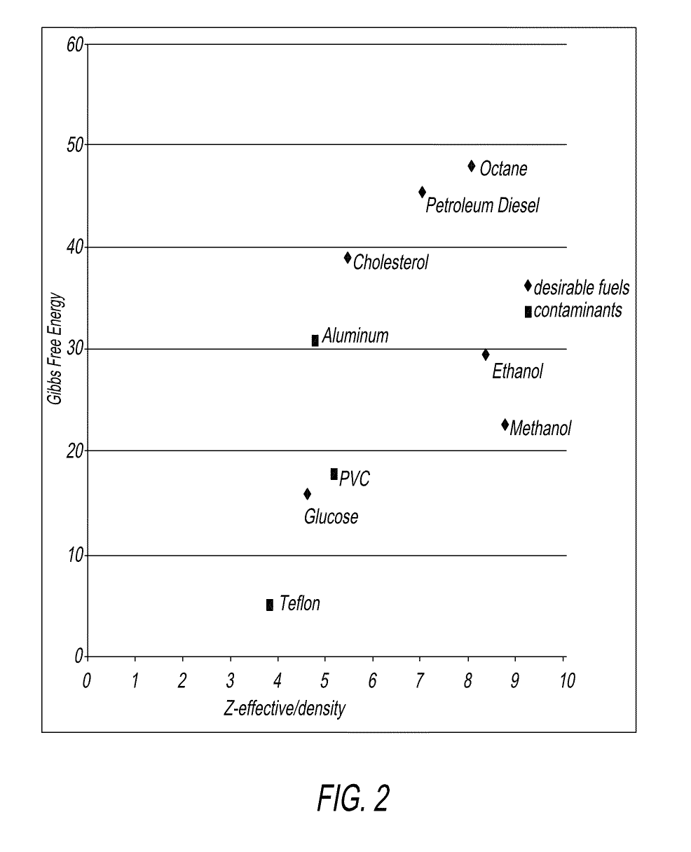

Example chart plotting the ratio of Z_{eff} to density (Z_{eff}/density) against Gibbs free energy for a number of materials is shown in FIG. 2. The value range requirement of Z_{eff} and density may be selected to provide a useful material having a relatively high Gibbs free energy, Helmholz free energy, higher heating value or lower heating value to carbon by weight ratio according to the plot. The ratio of Z_{eff} to density (Z_{eff}/density) in FIG. 2 also provides criteria to determine whether a high energetic material or a low energetic material may be added to the useful material during treating step prior to processing. For example, petroleum diesel has the ratio of Z_{eff} to density (Z_{eff}/density) greater than that for cholesterol but lower than octane; the low energetic material may be added during treating step prior to processing to producing energy. On the contrary, ethanol and methanol having the ratio of Z_{eff}/density higher than octane, the high energetic material may be added to during treating step. However, the criteria may change depending, for example, upon the specific material stream being treated, and the energy generation method being used.

One embodiment of the invention includes the use of a monitoring device such as, but not limited to, a dual-energy x-ray system. Dual-energy x-ray systems may be used to test a material for material parameters such as the effective atomic number (Z_{eff}) and the density of the material. The effective atomic number Z_{eff} (sometimes referred to as the effective nuclear charge) of an atom is the number of protons an electron in the element effectively “sees” due to screening by inner-shell electrons, and is a measure of the electrostatic interaction between the negatively charged electrons and positively charged protons in the atom. In one embodiment, dual-energy x-ray systems may be used to determine, with a significant degree of accuracy, the exact value of the Z_{eff} and density of the material stream being scanned.

One embodiment of the invention relates to a method of identifying and selecting a useful material (e.g., a useable fuel) from a stream of mixed material (e.g. a solid, liquid, and/or gas waste material stream) and segregating the fuel material from the residue. This useful fuel material may then be used to generate energy, while the residue may be disposed of safely or further processed. By separating out and using primarily fuel material that is efficient at generating energy, the efficiency of an energy generation system supplied with a mixed material waste stream can be greatly improved.

One embodiment of the method includes the steps of defining a value range requirement for at least one parameter of interest of fuel material to be selected from the mixed material stream. This value range requirement may, for example, include an upper and/or lower limit for one or more parameters of interest. Such parameters may include physical and chemical characteristics of the material being processed and may include, for example, Z_{eff} and/or density. In one embodiment, both Z_{eff} and density are used as the parameter of interest in the waste stream. In an alternative embodiment, other parameters may be used in addition to, or in place of, Z_{eff} and/or density. These parameters may include magnetic properties and/or chemical composition properties (e.g. acidity). Example parameters include, but are not limited to, the electric charge or current, and/or gravitational mass of materials under inspection (which may be measured, for example, by utilizing nuclear quadrupole resonance measurement systems), and/or the thickness, density and/or material composition of a material under inspection (which may be measured, for example, through THz-TDS (Terahertz time-domain spectroscopy)). THz-TDS may be particularly useful, for example, in measuring the amount of water contained in a sample.

Further parameters may include, but are not limited to, density and/or thickness information for the material under inspection (measured, for example, from Computed Tomography), and/or the rate at which slow neutrons return to a source after reflecting off of hydrogen nuclei contained in the material under inspection in order to determine the water content in the material and/or other atomic content of the material. In one embodiment, Ross filters and/or x-ray emitting diodes or lasers may be employed to radiate a material with specific energies of x-ray in order to develop a k-edge image of the material and identify and measure the amount of a single element that is present in the material—e.g. the carbon content. In one example embodiment, an x-ray emitting diode may be utilized in conjunction with an energy-sensitive x-ray detector in order to create a miniaturized energy-detection system that may be fitted into a fuel-line.

The values of the parameters or criteria of interest for a given portion of a material stream may be measured, for example by passing the material stream through one or more detectors, such as a dual-energy x-ray system. A dual-energy x-ray system is capable of readily measuring the Z_{eff} and the density of a material passing therethrough. The measured parameters (i.e., Z_{eff} and density) can then be compared to the predetermined ranges set for each of these parameters to determine whether the material portion passing through the detector falls within the defined ranges.

After the measured values for the material stream have been compared to the preset value range requirements for the parameters, the material stream may be separated, downstream of the detector, into fuel material and residue based on the measured parameter(s). More particularly, if the measured portion of material falls within the value range requirements (e.g., within the upper and lower limits set for both Z_{eff} and density), the material is designated as fuel material and continues along the fuel flow stream for further processing and energy extraction. However, if the measured portion of material falls outside the value range requirements (e.g., outside either an upper or lower limit for either or both of Z_{eff} and density), the material is designated as residue (or non-fuel material) and separated from the fuel flow stream, for example for further processing or disposal. In one embodiment, the measured parameters for the residue portion of the material stream may be used to further separate the residue, after separation from the fuel material, to collect the residue in one or more designated groups based on the type of residue. In one embodiment, the separation can be performed automatically, using air jets, chutes, or other means to direct or divert the fuel and residue components of the material stream.

In one embodiment, to enhance the exergetic efficiency of any real-world energy utilizing process, the methods described herein involve measurement of the Z_{eff} and density, determination of energetic content, measurement of the actual performance of the fuel, and feed the data back into any of a number of well-known machine learning algorithms (such as ANFIS—Adaptive Neuro Fuzzy Inference Systems), and making adjustments as to the fuzzy membership functions that define desirable fuel.

In one embodiment, the methods described herein may be used to develop sliding scales (e.g., fuzzy logic membership function scores) representing the “goodness” or grade of a mixed material stream, and/or a useful fuel material within a material stream, under inspection. This measure may be correlated—for example in a continuous function—for the energetic results from incineration, gasification and/or bio-digestion of the fuel. This information may be used to control, in part, the operation of the downstream energy production system.

One embodiment of the invention may further include processing the useful fuel material after separation but prior to producing energy from the fuel. This may be achieved, for example, by adding and mixing one or more treatment materials to the fuel, for example to adjust a fuel performance property of the fuel material. The treatment material may include, but is not limited to, high energetic material and low energetic material. For example, lignin (a chemical component of woody biomass) is relatively highly energetic and may be desirable as fuel. In one embodiment, a woody biomass material stream may be processed by selecting for wood chips that have high lignin content and eliminating wood chips with low lignin and high cellulose (a relatively low energy fuel), the overall energy density of the resulting fuel will be increased. In one embodiment, the treatment material to be added to the fuel is dependent upon the measured Z_{eff} and/or density of the fuel. As such, appropriate treatment materials may be added to each fuel portion to increase the efficiency and improve the performance of each fuel portion in generating energy. Alternatively, the measured Z_{eff} and/or density of the entire fuel material stream can be tallied, with the addition of treatment material occurring in a single step, based on the cumulative measured value of the fuel material.

In one example embodiment, low energetic material is added to the fuel when the ratio of Z_{eff} to density of the fuel material is greater than or equal to a first preset value, such as, but not limited to, a value of substantially the ratio of Z_{eff} to density for cholesterol (e.g. in a range of about 5-6) but less than a second value. In alternative embodiments, the first value, above which a low energetic material is added, may be any appropriate ratio (e.g. between about 1 and 7), depending, for example, upon the specific material stream being treated and the energy generation method being used, although, in alternative embodiments higher or lower values for the first preset value may be selected. In one embodiment high energetic material may be added to the fuel material when the ratio of Z_{eff} to density of the fuel material is greater than or equal to a second value, such as, but not limited to, a value of substantially the ratio of Z_{eff} to density for octane (e.g. in a range of about 7.5-8.5). As above, in alternative embodiments, the second value, above which a high energetic material is added, may be any appropriate ratio (e.g. between about 7 and 11 or higher) depending, for example, upon the specific material stream being treated, and the energy generation method being used, although, in alternative embodiments higher or lower values for the second preset value may be selected.

As a result, when the energetic content of the fuel stream under investigation is low, a “hotter” higher calorific value fuel may be added, in proportion to the energetic measure deficiency of the fuel, in order to deliver a fuel that will be maintain a gasification or incineration process at optimal performance levels. Similarly, if measurement of the parameters of the fuel determines that the fuel is more energetic than desired, a “cooling” lower calorific value fuel of known energy density may be added. As such, the fuel material can be optimized prior to processing to avoid operating the processing unit at suboptimal conditions (e.g., due to suboptimal fuel properties) that may be economically and environmentally costly.

In one embodiment, the treating step further includes measuring water content of the fuel material after separating the residue. The dual-energy transmissivity of the fuel material may then be determined, and an adjusted dual-energy transmissivity calculated by compensating for the measured water content in the fuel material. The type and quantity of treatment material (e.g., high energetic material and/or low energetic material) to be added may then be adjusted to compensate for water content, thereby further improving the quality of the fuel material. The water content may be measured, for example, using methods based on, but not limited to, microwave, mm-wave, and/or THz technology.

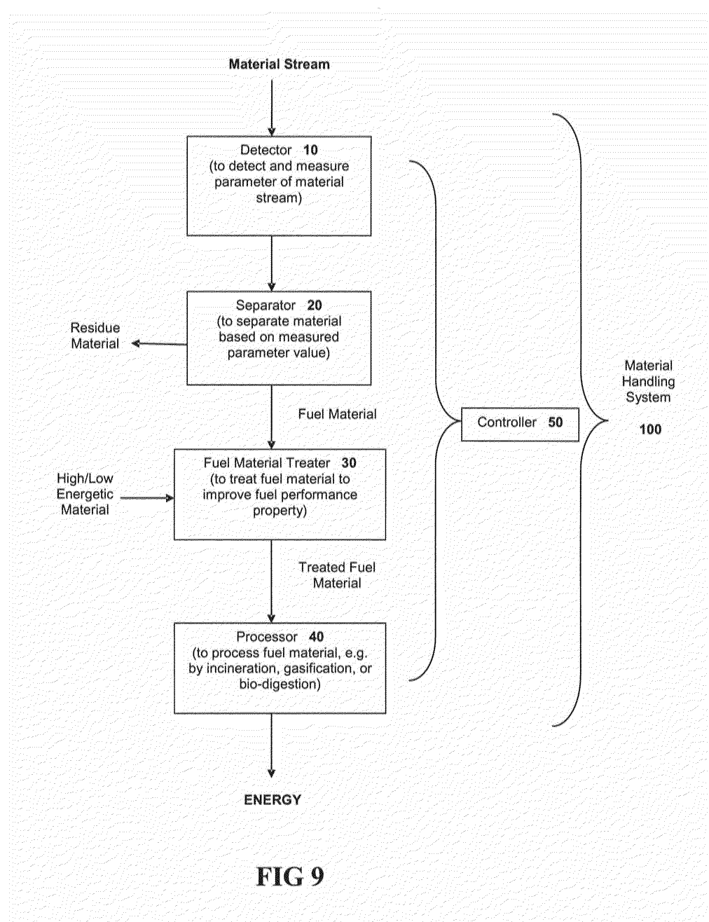

An example material handling system for detecting and separating desirable material (e.g., fuel) from undesirable waste material in a material stream is shown in FIG. 9. In this embodiment, the material stream (e.g., including mixed solid, liquid, and/or gaseous materials) passes through a Detector 10 (e.g., a dual-energy x-ray) to detect, analyze, and measure a value of at least one parameter of the material within the material stream. The material stream is then passed through a Separator 20 to separate the desirable fuel material from undesirable waste/residue material based on the at least one measured parameter value. The Separator 20 may separate the fuel material from the waste material through any appropriate means including, but not limited to, mechanical means (e.g., trap doors, pushers, collection arms, etc) and/or blowers and/or suction elements.

After separating the waste material from the fuel material, the fuel material may be passed through a Treater 30 to treat the fuel material (e.g., by adding a high or low energetic material) to improve a fuel performance property of the fuel material. In an alternative embodiment, the Treater 30 may not be required. The fuel material is then passed to one or more Processors 40 for processing the fuel material to produce energy through, for example, incineration, gasification, or bio-digestion.

One or more Controllers 50 may be connected to one or more of the system elements to control at least a portion of the process. For example, a single Controller may be used to control the passage of the material stream through the system, analyze measured data from the Detector 10, control the separation of the material in the Separator 20 based on the analyzed data, and control the treatment of the fuel material in the Treater 30. The Controller 50 may be used, for example, to automate the material handling system 100, or portions thereof. The Controller 50 may include an analyzer for analyzing the data received from the detector 10 and designating the material passing through the Detector 10 as either fuel or waste based on the measured parameter value. The Controller 50 may include one or more user interface elements to allow user input (e.g., of parameter value range requirements) and user control of the system, and to provide a user with output information related to the operation of the system (e.g., measured data from the material stream, ratio of fuel to waste, warning signals, etc). In one embodiment, multiple Controllers 50 may be used to control various stages of the material handling and/or other components of the overall system 100.

The material stream may be transported through the system using a transporting means such as, but not limited to, a conveyer belt, pipeline, and/or a gravity driven channel.

Example methods for selecting and processing a fuel material from a mixed material stream are described below.

### Example 1

In this example, the method includes passing a material stream through a dual-energy x-ray and measuring Z_{eff} and density of the material stream. If Z_{eff} is greater than 8, do not select for fuel, and designate as residue. If density is equal to or greater than the density of PVC, then do not select for fuel, and designate as residue. If the measured material stream portion has both a Z_{eff} of less than 8, and a density of less than that of PVC, designate as fuel. After measuring, separate the material stream into a fuel stream material portion and a residue material portion.

### Example 2

In this example, once the fuel portion has been identified and separated from the residue, the quality of the fuel can be optimized through addition of a treatment material (e.g., high energetic material and/or low energetic material). In operation, if Z_{eff}/density is equal to or less than the ratio for PVC, then do not use for fuel. If Z_{eff}/density is equal to or greater than the ratio for cholesterol but lower than the ratio for octane, add a “cooling,” low energetic fuel to the mix to optimize performance, and if Z_{eff}/density is equal to or greater than the ratio for octane, add a “warming,” high energetic fuel to optimize performance. However, the criteria may change depending, for example, upon the specific material stream being treated, and the energy generation method being used.

### Example 3

In this example, the quality of the fuel portion may further be improved by compensating for water content, for example by incorporating a measurement of the water content of the fuel material under inspection from an orthogonal technology and use that information to better determine density and Z_{eff} of the non-water materials in order to optimize the fuel (and the water content) for the performance of the energy conversion technology. In operation, measure the water content using microwave, mm-wave or THz technology. Measure the dual-energy transmissivity of the material, and use the water content to mathematically eliminate the contribution of the water in the sample to the dual-energy transmissivity. These “water-content adjusted” results may then be used in the quality optimization process described above, for example, in Example 2. According to one technique, the fuel may be treated with wetter or drier fuel of known energetic content in order to optimize for both the energetic content and the water content for use in an incineration, gasification or other method of using the fuel.

### Example 4

In this example, the locus of a material defines the center of a fuzzy-logic membership function. According to one technique, sugar (e.g., glucose) may be set as an appropriate analog for a desirable fuel, with salt an appropriate analog for an undesirable fuel. By taking average H and average L (where H is the log of the attenuation of the voxel under inspection when illuminated by high energy x-ray and L is the equivalent low energy x-ray image) or a sample of two test fuel materials, and comparing these results against salt and glucose, the desirability of a test material as a fuel can be determined. In one embodiment, the high energy x-ray may be approximately 90 keV, while the low energy x-ray may be approximately 60 keV. More particularly, the degree to which each pixel in an image is similar to the [H, L] vector for salt would be the degree to which the material represented by the pixel corresponded to a collection of inorganic materials. The degree to which the pixel was similar to the [H, L] vector for sugar would represent the degree to which the material represented by the pixel would correspond to a collection of organic materials.

As the samples used in this example were from a woody material salvaged from construction and demolition waste, the sugar-salt dichotomy provided an appropriate comparison test, as woody materials that are more similar to sugar have more lignin, and the more lignin, the more desirable the woody material would be as a fuel.

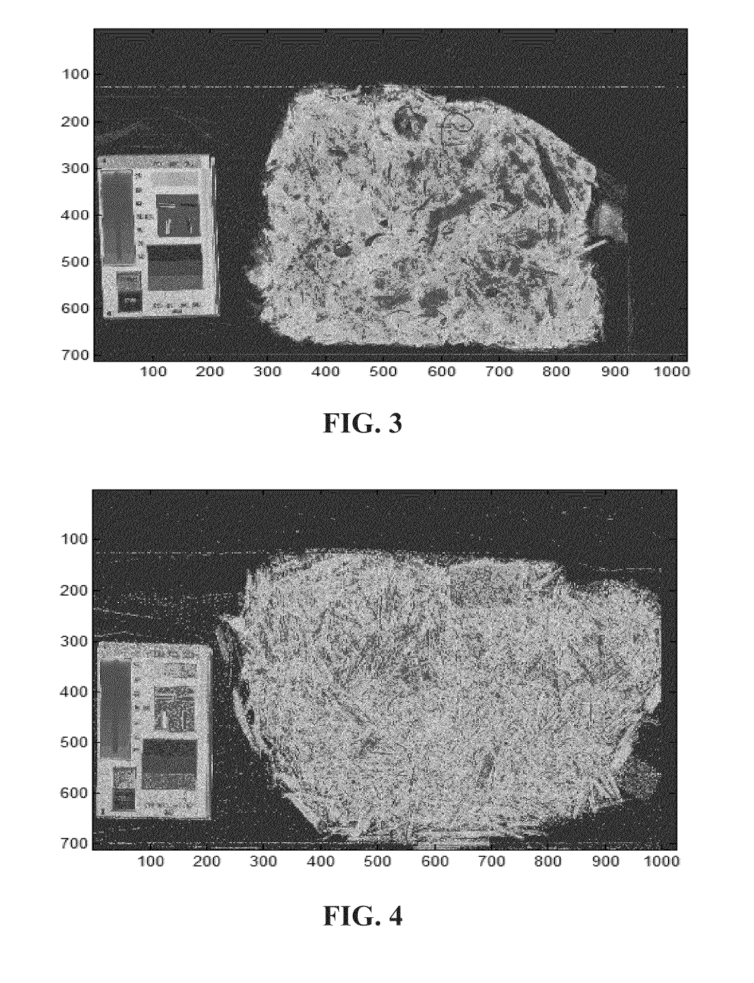

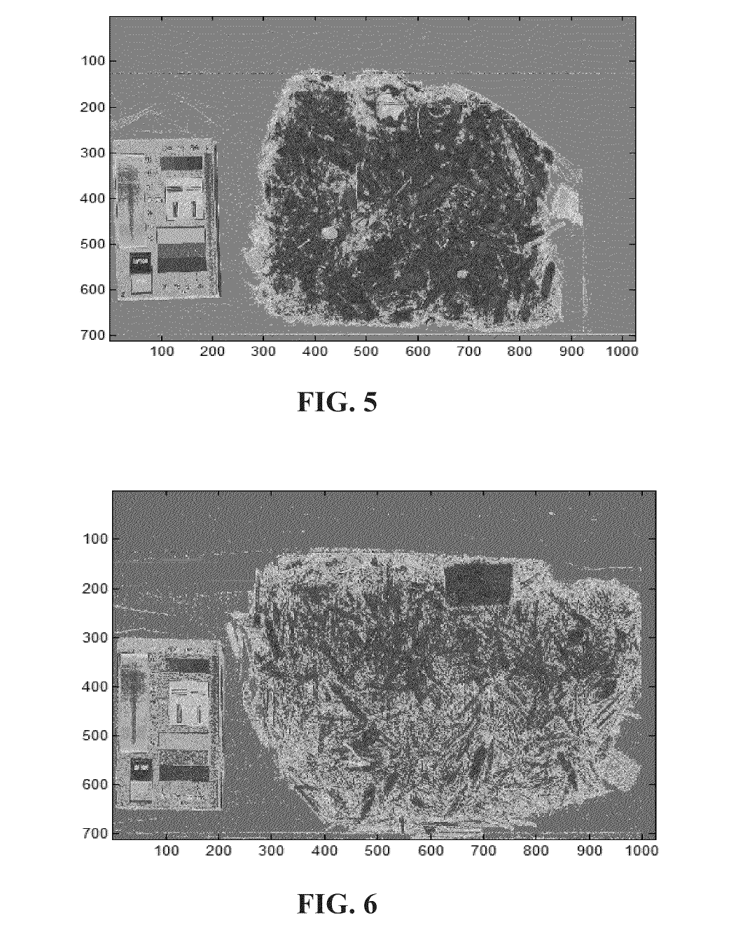

For a computationally efficient measure of each pixel's membership in the sugar and salt fuzzy sets, a dot-product method was used. Each pixel had an [H, L] component. A dot product was produced for each pixel against the (1) sugar and (2) salt representative vectors. Example dot-product results for two tested fuel materials against salt are shown in FIGS. 3 and 4, while example dot-product results for two tested fuel materials against sugar are shown in FIGS. 5 and 6. The inorganic content of the two sample materials is represented by the salt image (an image of the dot-product of each pixel's [H, L] vector with the [H, L] vector of salt).

The results show that metallic items may be identified and separated (e.g. a wire in the material of FIG. 3 and arsenic impregnation of the wooden material of FIG. 4). The sugar images of FIGS. 5 and 6 show a clear difference between the two materials as for their similarity to sugar. This similarity to sugar corresponded to a known difference in energetic value of the two sample fuels.

### Example 5

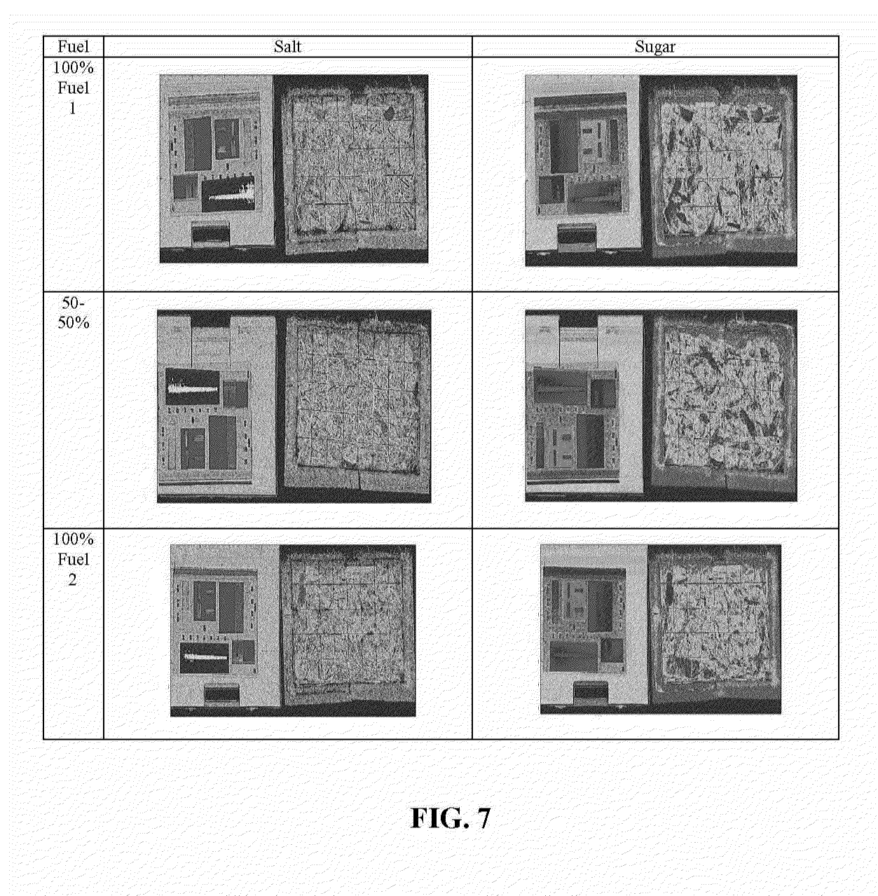

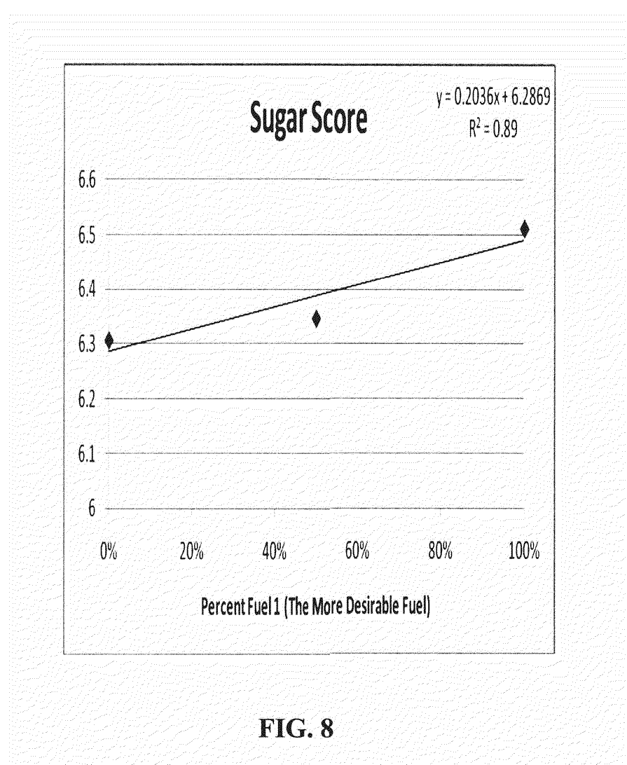

In order to determine that the energetic estimates are not simply the result of differences in mass or density of the two sets of material, a second experiment using a box with a fixed area and an open top was performed. The same mass of material (1 kg) was placed in the box for three experimental runs. In one run, it contained 100% of a first fuel, then 50:50% of a first and second fuel, then 100% of the second fuel. The images were processed as above. Then, the fuzzy score for each pixel in the membership function of salt and sugar were summed up, and then an exergetic analog score was obtained. The resulting comparison pixel charts as a function of salt and sugar are shown in FIG. 7. A chart summarizing the different sugar scores as a function of the percentage of the first fuel is shown in FIG. 8.

This ability to differentiate, given the rough nature of an analysis based solely on H and L, shows that a similar analysis run with density and Z_{eff}-based fuzzy sets (or, in one embodiment, water-corrected Z_{eff} and density-based fuzzy sets) allows the methods described herein to produce a continuous grade for fuel for use in incinerators, gasifiers and bio-digestion systems.

It should be understood that alternative embodiments, and/or materials used in the construction of embodiments, or alternative embodiments, are applicable to all other embodiments described herein.

The invention may be embodied in other specific forms without departing from the spirit or essential characteristics thereof. The foregoing embodiments, therefore, are to be considered in all respects illustrative rather than limiting the invention described herein. Scope of the invention is thus indicated by the appended claims, rather than solely by the foregoing description, and all changes that come within the meaning and range of equivalency of the claims are intended to be embraced therein. Additional scope of the invention may be found in any disclosed, but unclaimed, subject matter described herein.

While there have been shown and described and pointed out the fundamental novel features of the invention as applied to the preferred embodiments, it will be understood that the foregoing is considered as illustrative only of the principles of the invention and not intended to be exhaustive or to limit the invention to the precise forms disclosed. Obvious modifications or variations are possible in light of the above teachings. The embodiments discussed were chosen and described to provide the best illustration of the principles of the invention and its practical application to enable one of ordinary skill in the art to utilize the invention in various embodiments and with various modifications as are suited to the particular use contemplated All such modifications and variations are within the scope of the invention as determined by the appended claims when interpreted in accordance with the breadth to which they are entitled.

What is claimed is:

1. A method of selecting useful material from a mixed solid, liquid or gaseous material stream, the method comprising the steps of: defining a value range requirement for at least one parameter of interest of useful material to be selected from the material stream; passing the material stream through at least one detector adapted to measure the parameter of interest of the material stream; and separating the material stream into useful material and residue based on the measured parameter.

2. The method of claim 1, wherein the parameter of interest comprises at least one of Z_{eff} and density.

3. The method of claim 2, wherein an upper limit for density comprises density of PVCs and an upper limit for Z_{eff} is 8.

4. The method of claim 1, wherein the at least one detector is adapted to measure at least one of Z_{eff} and density of the material stream.

5. The method of claim 2, wherein a value range requirement of Z_{eff} and density is selected to provide a useful material having a relatively high Gibbs free energy, Helmholz free energy, Higher Heating Value or Lower Heating Value to carbon by weight ratio.

6. The method of claim 1, further comprising processing the useful material to produce energy; wherein the processing step comprises at least one of incineration, gasification, and bio-digestion.

7. The method of claim 6, further comprising, prior to processing, treating the useful material to adjust a fuel performance property of the useful material; wherein the treating step comprises at least one of adding a high energetic material and low energetic material to the useful material.

8. The method of claim 7, wherein the low energetic material is added when a ratio of Z_{eff} to density of the useful material is greater than or equal to a first value but less than a second value; wherein the high energetic material is added when a ratio of Z_{eff} to density of the useful material is greater than or equal to a second value.

9. The method of claim 8, wherein the first value comprises a value of a ratio of Z_{eff} to density for cholesterol; wherein the second value comprises a value of a ratio of Z_{eff} to density for octane.

10. The method of claim 7, wherein the treating step comprises: measuring a water content of the useful material; measuring a dual-energy transmissivity of the useful material; calculating an adjusted dual-energy transmissivity by compensating for the measured water content; and adjusting addition of high-energetic material and low-energetic material based at least in part thereon; wherein the water content is measured using a method selected from the group consisting of microwave, mm-wave, and THz technology.

11. The method of claim 1, further comprising treating the residue based on a parameter of the residue measured by the detector; wherein the parameter comprises at least one of Z_{eff} and density.

12. A system for selecting a useful material from a mixed solid, liquid or gaseous material stream passing through the system comprising: at least one detector adapted to measure a parameter of interest of the material stream passing therethrough; and at least one separator for separating the material stream into useful material and residue based on the measured parameter and a value range requirement after passing through the detector.

13. The system of claim 12, wherein the at least one detector is adapted to measure at least one of Z_{eff} and density of the material stream.

14. The system of claim 12, wherein the at least one detector comprises a dual-energy x-ray.

15. The system of claim 12, further comprising a processor for processing the useful material to produce energy; wherein the processor comprises at least one of an incinerator, a gasifier, and a bio-digestor.

16. The system of claim 15, further comprising at least one treater for treating the useful material to adjust a fuel performance property of the useful material prior to processing; wherein the treater adds at least one of a high energetic material and a low energetic material to the useful material.

17. The system of claim 16, further comprising at least one controller for controlling operation of at least one of the system, the detector, the separator, the treater, and the processor.

18. The method of claim 1 further comprises comparing the high energy x-ray (H) and low energy x-ray (L) results against salt and glucose to determine the desirability of a material; the degree to which each pixel in an image is similar to the [H, L] vector for sugar represents the degree to which the material represented by the pixel corresponds to a collection of organic materials; and the degree to which each pixel in an image is similar to the [H, L] vector for salt represents the degree to which the material represented by the pixel corresponds to a collection of inorganic materials.

19. A method for enhancing the exergetic efficiency comprises measuring the Z_{eff}; measuring density; determining energy content; measuring actual performance of a fuel; feeding data of Z_{eff}, density, energy content into at least one of algorithms; and making adjustments as to the membership functions that define desirable fuel.

20. The method of claim 1, wherein the method can be used to grade algal fuel and selecting individual organisms in the algal culture that have superior energy storing performance.
